(19) United States
(12) Patent Application Publication
(10) Pub. No.: US 2012/0149678 A1
(43) Pub. Date: Jun. 14, 2012

(54) Organonitro Compounds for Use in Treating Non-Hodgkin's Lymphoma and Leukemia, and Methods Relating Thereto

(76) Inventors: Bryan T. Oronsky, Los Altos Hills, CA (US); Susan Knox, Stanford, CA (US); Jan Scicinski, Saratoga, CA (US); Shoucheng Ning, Santa Clara, CA (US)

(21) Appl. No.: 13/315,888

(22) Filed: Dec. 9, 2011

Related U.S. Application Data
(60) Provisional application No. 61/421,337, filed on Dec. 9, 2010.

(51) Int. Cl. A61K 31/397; A61K 31/4468; A61K 31/12; A61K 31/16; A61K 31/04; A61P 35/02; A61K 31/445; A61K 31/255; A61K 31/22; A61K 31/40; A61P 35/00

(52) U.S. Cl. 514/210.17; 514/423; 514/315; 514/676; 514/210.01; 514/426; 514/628; 514/517; 514/518; 514/550; 514/742; 514/740

(57) ABSTRACT

The invention provides organonitro compounds, compositions containing such compounds, medical kits, and methods for using such compounds and compositions to treat non-Hodgkin's lymphoma and certain leukemias in a patient. The compounds, compositions, kits, and methods are contemplated to provide a therapeutic benefit in treating non-Hodgkin's lymphoma, chronic lymphocytic leukemia, acute myelogenous leukemia, chronic myelogenous leukemia, and acute lymphoblastic leukemia. Exemplary organonitro compounds described herein include alkyl-substituted and acyl-substituted di-nitroheterocycles.

### RELATED APPLICATIONS

This application claims the benefit of and priority to U.S. Provisional Patent Application Ser. No. 61/421,337, filed Dec. 9, 2010, the contents of which are hereby incorporated by reference.

### FIELD OF THE INVENTION

The invention provides organonitro compounds, compositions containing such compounds, medical kits, and methods for using such compounds and compositions to treat non-Hodgkin's lymphoma and certain leukemias in a patient.

### BACKGROUND

Cancer is a leading cause of death worldwide. Approximately one million people are diagnosed with cancer each year in the United States, and approximately half a million cancer patients die annually despite the significant progress made during the last decade in both the diagnosis and treatment of cancer.

Non-Hodgkin's lymphoma is a type of cancer derived from lymphocytes. Various reports describe treating patients with non-Hodgkin's lymphoma using chemotherapy, immunotherapy, radiation, hematopoietic stem cell transplantation, and/or monoclonal antibodies. The severity of non-Hodgkin's lymphoma can vary from indolent to very aggressive.

Leukemia is a cancer of the bone marrow or blood that is characterized by an abnormal increase in white blood cells. There are multiple types of leukemia, which can be characterized according to whether the leukemia is chronic or acute, and according to the type of blood cell affected. Chronic lymphocytic leukemia (CLL) affects mostly adult men, and has a 75% five-year survival rate. Acute myelogenous leukemia (AML) also affects mostly adult men, is often treated with chemotherapy, and has a 45% five-year survival rate. Chronic myelogenous leukemia (CML) affects mostly adults, is often treated with drugs, such as imatinib, and has a 90% five-year survival rate. Acute lymphoblastic leukemia (ALL) is the most common type of leukemia in young children and is typically treated with chemotherapy and radiation. Survival rates for patients suffering from ALL are 85% in children and 50% in adults.

Because of the inadequate patent survival rate for current therapies for treating Non-Hodgkin's lymphoma and certain forms of leukemia, the need exists for new therapeutic methods for treating these disorders. The present invention fulfills this need and provides other related advantages.

### SUMMARY

The invention provides organonitro compounds, compositions containing such compounds, medical kits, and methods for using such compounds and compositions to treat non-Hodgkin's lymphoma and certain leukemias in a patient. Various aspects and embodiments of the invention are described in further detail below.

Accordingly, one aspect of the invention provides a family of organonitro compounds embraced by Formula I for use in the methods, compositions and kits described herein, wherein Formula I is represented by:

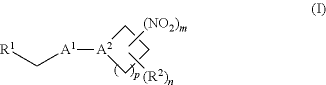

or a pharmaceutically acceptable salt or solvate thereof, wherein the variables are as defined in the detailed description.

Another aspect of the invention provides a family of organonitro compounds embraced by Formula II for use in the methods, compositions and kits described herein, wherein Formula II is represented by:

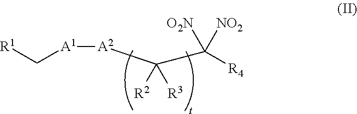

or a pharmaceutically acceptable salt or solvate thereof, wherein the variables are as defined in the detailed description.

Another aspect of the invention provides a method of treating a disorder selected from the group consisting of non-Hodgkin's lymphoma, chronic lymphocytic leukemia, acute myelogenous leukemia, chronic myelogenous leukemia, and acute lymphoblastic leukemia. The method comprises administering to a patient in need thereof a therapeutically effective amount of an organonitro compound described herein, such as a compound of Formula I or II.

Another aspect of the invention provides a kit for treating a disorder. The kit comprises i) instructions for treating a disorder selected from the group consisting of non-Hodgkin's lymphoma, chronic lymphocytic leukemia, acute myelogenous leukemia, chronic myelogenous leukemia, and acute lymphoblastic leukemia; and ii) an organonitro compound described herein, such as a compound of Formula I or II.

### BRIEF DESCRIPTION OF THE FIGURES

FIG. 1 is a graph showing the percentage of B-cell lymphoma Daudi cells that underwent apoptosis after exposure to 1-bromoacetyl-3,3-dinitroazetidine (ABDNAZ).

FIG. 2 is a graph showing the percentage of B-cell lymphoma Daudi cells (which were treated with a glutathione (GSH) precursor and a GSH synthesis inhibitor) that underwent apoptosis after exposure to 1-bromoacetyl-3,3-dinitroazetidine (ABDNAZ).

### DETAILED DESCRIPTION OF THE INVENTION

The invention provides organonitro compounds, compositions containing such compounds, kits, and methods for using such compounds and compositions to treat non-Hodgkin's lymphoma and certain leukemias in a patient. The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. DEFINITIONS

To facilitate an understanding of the present invention, a number of terms and phrases are defined below.

The terms “a” and “an” as used herein mean “one or more” and include the plural unless the context is inappropriate.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc

The term “haloalkyl” refers to an alkyl group that is substituted with at least one halogen. For example, —CH₂F, —CHF₂, —CF₃, —CH₂CF₃, —CF₂CF₃, and the like.

The term “aralkyl” refers to an alkyl group substituted with an aryl group.

The term “heteroaralkyl” refers to an alkyl group substituted with a heteroaryl group.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with halogen, alkyl, hydroxyl, or alkoxyl. The term “aryl” also includes polycyclic ring systems having two or more carbocyclic rings in which two or more carbons are common to two adjoining rings (the rings are “fused rings”) wherein at least one of the rings is aromatic, and, e.g., the other cyclic ring(s) may be cycloalkyls, cycloalkenyls, cycloalkynyls, and/or aryls.

The “heteroaryl” is art-recognized and refers to aromatic groups that include at least one ring heteroatom. In certain instances, a heteroaryl group contains 1, 2, 3, or 4 ring heteroatoms. Representative examples of heteroaryl groups includes pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with halogen, alkyl, hydroxyl, or alkoxyl. The term “heteroaryl” also includes polycyclic ring systems having two or more rings in which two or more carbons are common to two adjoining rings (the rings are “fused rings”) wherein at least one of the rings is heteroaromatic, and, e.g., the other cyclic rings may be cycloalkyl(s), cycloalkenyls, cycloalkynyls, and/or aryls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

As used herein, the term “heterocyclic” represents, for example, an aromatic or nonaromatic ring containing one or more heteroatoms. The heteroatoms can be the same or different from each other. Examples of heteroatoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above.

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans. The term “non-anemic patient” refers to a patient that does not suffer from anemia.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. ORGANONITRO COMPOUNDS FOR USE IN THE METHODS, COMPOSITIONS, AND KITS DESCRIBED HEREIN

One aspect of the invention provides organonitro compounds for use in the methods, compositions and kits described herein. In certain embodiments, the organonitro compound is a compound embraced by Formula I:

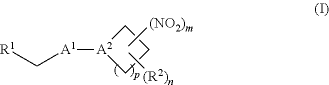

or a pharmaceutically acceptable salt or solvate thereof, wherein:

A¹ is —C(O)— or —(C(R³)₂)—C(O)(C(R³)₂)ₓ—;

A² is N or —C(R⁴)—;

R¹ is halogen, —OS(O)₂R⁵, or —OC(O)CF₃;

R² is C₁-C₆alkyl;

R³ and R⁴ each represent independently for each occurrence hydrogen or C₁-C₅alkyl;

R⁵ is C₁-C₆alkyl, C₁-C₆haloalkyl, aryl, or aralkyl;

m and p are independently 1, 2, or 3; and

n and x each represent independently for each occurrence 0, 1, 2, or 3.

In certain embodiments, A¹ is —C(O)—. In certain other embodiments, A¹ is —(C(R³)₂)ₓC(O)(C(R³)₂)ₓ—. In certain other embodiments, A¹ is —C(O)(C(R³)₂)ₓ—.

In certain embodiments, A² is N. In certain other embodiments, A² is —C(R⁴)—.

In certain embodiments, R¹ is halogen, —OS(O)₂R⁵, or —OC(O)CF₃. In certain other embodiments, R¹ is halogen. In certain other embodiments, R¹ is —OS(O)₂R⁵. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain other embodiments, R¹ is bromo.

In certain embodiments, m is 2. In certain other embodiments, m is 1.

In certain embodiments, n is 0. In certain other embodiments, n is 1. In certain other embodiments, n is 2.

In certain embodiments, p is 1. In certain other embodiments, p is 2. In certain other embodiments, p is 3.

The description above describes multiple embodiments relating to compounds of Formula I. The patent application specifically contemplates all combinations of the embodiments. For example, the invention contemplates a compound of Formula I wherein A¹ is —C(O)—, A² is N, R¹ is halogen, and n is 0.

In certain embodiments, the compound is a compound of Formula I-A:

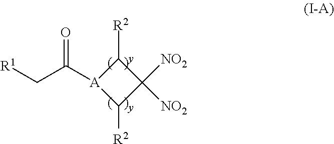

or a pharmaceutically acceptable salt or solvate thereof, wherein:
A is N or C(H);
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R² represents independently for each occurrence hydrogen or methyl; and
y represents independently for each occurrence 1 or 2.

In certain embodiments, A is N. In certain other embodiments, A is C(H).

In certain embodiments, R¹ is chloro or bromo. In certain embodiments, R¹ is chloro. In certain other embodiments, R¹ is bromo. In certain other embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² is hydrogen or methyl. In certain other embodiments, R² is hydrogen.

In certain embodiments, y is 1. In certain other embodiments, one occurrence of y is 1, and the other occurrence of y is 2. In certain other embodiments, y is 2.

The description above describes multiple embodiments relating to compounds of Formula I-A. The patent application specifically contemplates all combinations of the embodiments. For example, the invention contemplates a compound of Formula I-A wherein A is N, R¹ is chloro or bromo, and R² is hydrogen.

In certain embodiments, the compound is

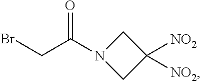

or a pharmaceutically acceptable salt or solvate thereof. In certain other embodiments, the compound is

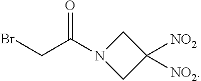

In certain other embodiments, the organonitro compound is a compound embraced by Formula II:

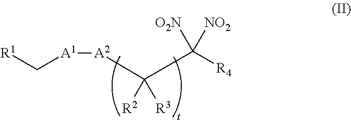

or a pharmaceutically acceptable salt or solvate thereof: wherein:

A¹ is —C(O)— or —(C(R⁵)₂)ₓC(O)(C(R⁵)₂)ₓ—;

A² is —N(R⁵)— or —C(R²)(R³)—;

R¹ is halogen, —OS(O)₂R⁶, or —OC(O)CF₃;

R² and R³ each represent independently for each occurrence hydrogen or C₁-C₆alkyl; or R² and R³ are taken together with the carbon atom to which they are attached to form a 3-6 membered, saturated carbocyclic ring;

R⁴ is hydrogen or C₁-C₆alkyl;

R⁵ represents independently for each occurrence hydrogen or C₁-C₆alkyl;

R⁶ is C₁-C₆alkyl, C₁-C₆haloalkyl, aryl, or aralkyl;

t is an integer in the range from 1 to 12; and

x represents independently for each occurrence 0, 1, 2, or 3.

In certain embodiments, A¹ is —C(O)—. In certain other embodiments, A¹ is —(C(R⁵)₂)ₓC(O)(C(R⁵)₂)ₓ—. In certain other embodiments, A¹ is —C(O)(C(R⁵)₂)ₓ—.

In certain embodiments, A² is —N(R⁵)—. In certain other embodiments, A² is —C(R²)(R³)—.

In certain embodiments, R¹ is halogen. In certain other embodiments, R¹ is —OS(O)₂R⁶. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain embodiments, R¹ is bromo.

In certain embodiments, R² and R³ each represent independently for each occurrence hydrogen or C₁-C₆alkyl. In certain other embodiments, R² and R³ each represent independently for each occurrence hydrogen, methyl, ethyl, or propyl. In certain other embodiments, R² and R³ each represent independently for each occurrence hydrogen or methyl. In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

In certain embodiments, R⁶ is C₁-C₆alkyl or C₁-C₆haloalkyl. In certain other embodiments, R⁶ is methyl, ethyl, or trifluoromethyl. In certain other embodiments, R⁶ is aryl, such as phenyl.

In certain embodiments, t is 1, 2, 3, 4, 5 or 6. In certain other embodiments, t is 1, 2, or 3. In certain other embodiments, t is 1. In certain embodiments, x is 1 or 2.

The description above describes multiple embodiments relating to compounds of Formula II. The patent application specifically contemplates all combinations of the embodiments. For example, the invention contemplates a compound of Formula II wherein A¹ is —C(O)—, A² is —N(R⁵)—, and R² and R³ are hydrogen.

In certain embodiments, the compound is a compound of Formula II-A:

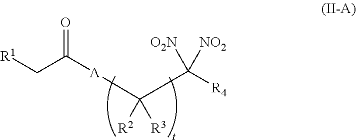

or a pharmaceutically acceptable salt or solvate thereof: wherein:

A is —N(R⁵)— or —C(R²)(R³)—;

R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;

R², R³, and R⁵ each represent independently for each occurrence hydrogen or methyl;

R⁴ is hydrogen or C₁-C₆alkyl; and

t is 1, 2, or 3.

In certain embodiments, A is —N(R⁵)—. In certain other embodiments, A is —N(CH₃)—. In certain other embodiments, A is —C(R²)(R³)—. In certain other embodiments, A is —CH₂—.

In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

The description above describes multiple embodiments relating to compounds of Formula II-A. The patent application specifically contemplates all combinations of the embodiments. For example, the invention contemplates a compound of Formula II-A wherein A is —N(R⁵)—, and R² and R³ are hydrogen.

In certain other embodiments, the compound is one of the compounds listed in Tables 1, 2, or 3 below or a pharmaceutically acceptable salt or solvate thereof.

TABLE 1

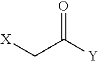

| Compound | | |
|---|---|---|
| No. | X | Y |
| I-1 | Br | 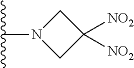 |
| I-2 | Br | 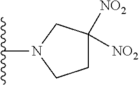 |
| I-3 | Br | 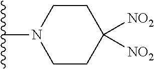 |
| I-4 | Br | 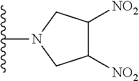 |
| I-5 | Br | 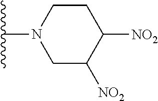 |
| I-6 | Br | 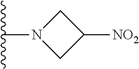 |
| I-7 | Br | 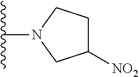 |
| I-8 | Br | 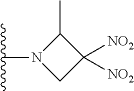 |
| I-9 | Br | 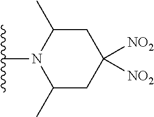 |
| I-10 | Br | 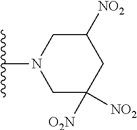 |
| I-11 | Br | 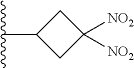 |
| I-12 | Br | 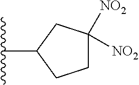 |
| I-13 | Br | 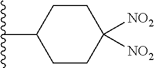 |
| I-14 | Br | 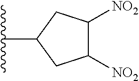 |
| I-15 | Br | 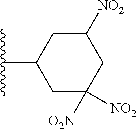 |
| I-16 | Br | 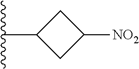 |
| I-17 | Br | 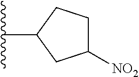 |
| I-18 | Br | 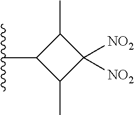 |
| I-19 | Cl | 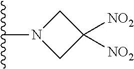 |
| I-20 | Cl | 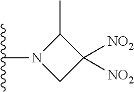 |
| I-21 | Cl | 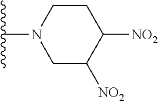 |
| I-22 | Cl | 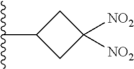 |
| I-23 | I | 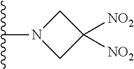 |
| I-24 | I | 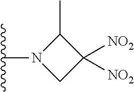 |
| I-25 | I | 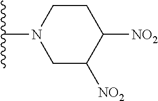 |
| I-26 | I | 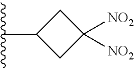 |
| I-27 | —OS(O)₂CH₃ | 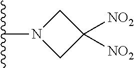 |
| I-28 | —OS(O)₂CH₃ | 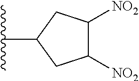 |
| I-29 | —OS(O)₂CF₃ | 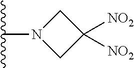 |
| I-30 | —OS(O)₂CF₃ | 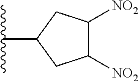 |
| I-31 | 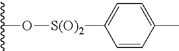 | 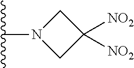 |
| I-32 | 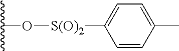 | 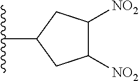 |
| I-33 | —OC(O)CF₃ | 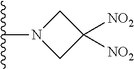 |
| I-34 | —OC(O)CF₃ | 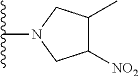 |
| I-35 | —OC(O)CF₃ | 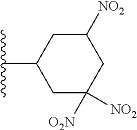 |

TABLE 2

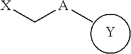

| Compound No. | X | A | Y |
|---|---|---|---|
| II-1 | Br | —CH₂C(O)— | 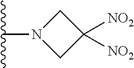 |
| II-2 | Br | —CH₂C(O)— | 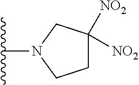 |
| II-3 | Br | —CH₂C(O)— | 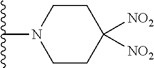 |
| II-4 | Br | —CH₂C(O)— | 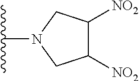 |
| II-5 | Br | —CH₂C(O)— | 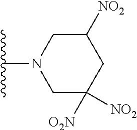 |
| II-6 | Br | —CH₂C(O)— | 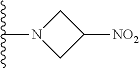 |
| II-7 | Br | —CH₂C(O)— | 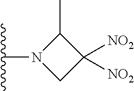 |
| II-8 | Br | —C(O)CH₂CH₂— | 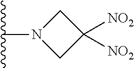 |
| II-9 | Br | —C(O)CH₂CH₂— | 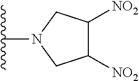 |
| II-10 | Br | —C(O)CH₂CH₂— | 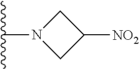 |
| II-11 | Br | —C(O)CH₂CH₂— | 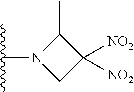 |
| II-12 | Br | —CH₂— | 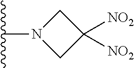 |
| II-13 | Br | —CH₂— | 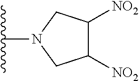 |
| II-14 | Br | —CH₂— | 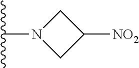 |
| II-15 | Br | —CH₂— | 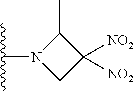 |
| II-16 | Br | —CH₂C(O)— | 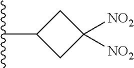 |
| II-17 | Br | —CH₂C(O)— | 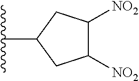 |
| II-18 | Br | —C(O)CH₂CH₂— | 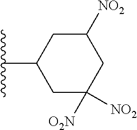 |
| II-19 | Br | —C(O)CH₂CH₂— | 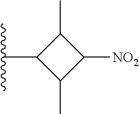 |
| II-20 | Br | —CH₂— | 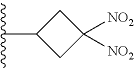 |
| II-21 | Br | —CH₂— | 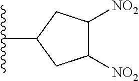 |
| II-22 | Br | —CH₂— | 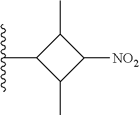 |
| II-23 | Cl | —CH₂C(O)— | 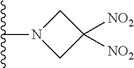 |
| II-24 | Cl | —C(O)CH₂CH₂— | 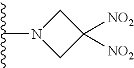 |
| II-25 | Cl | —CH₂— | 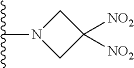 |
| II-26 | Cl | —CH₂C(O)— | 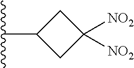 |
| II-27 | Cl | —C(O)CH₂CH₂— | 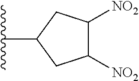 |
| II-28 | Cl | —CH₂— | 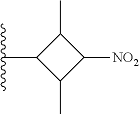 |
| II-29 | —OS(O)₂CH₃ | —CH₂C(O)— | 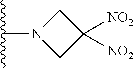 |
| II-30 | —OS(O)₂CH₃ | —CH₂C(O)— | 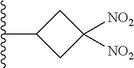 |
| II-31 | —OS(O)₂CH₃ | —C(O)CH₂CH₂— | 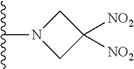 |
| II-32 | —OS(O)₂CH₃ | —CH₂— | 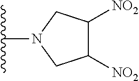 |
| II-33 | —OS(O)₂CF₃ | —CH₂C(O)— | 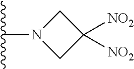 |
| II-34 | —OS(O)₂CF₃ | —CH₂C(O)— | 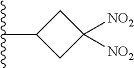 |
| II-35 | —OS(O)₂CF₃ | —C(O)CH₂CH₂— | 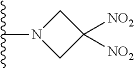 |
| II-36 | —OS(O)₂CF₃ | —CH₂— | 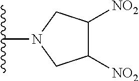 |
| II-37 | 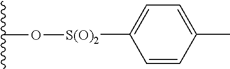 | —CH₂C(O)— | 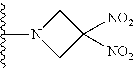 |
| II-38 | 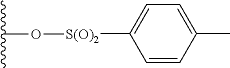 | —CH₂C(O)— | 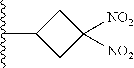 |
| II-39 | 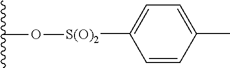 | —C(O)CH₂CH₂— | 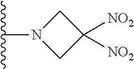 |
| II-40 | 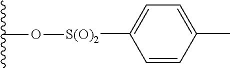 | —CH₂— | 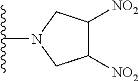 |
| II-41 | —OC(O)CF₃ | —CH₂C(O)— | 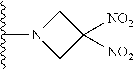 |
| II-42 | —OC(O)CF₃ | —CH₂C(O)— | 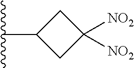 |
| II-43 | —OC(O)CF₃ | —C(O)CH₂CH₂— | 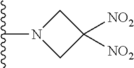 |
| II-44 | —OC(O)CF₃ | —CH₂— | 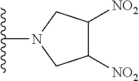 |

TABLE 3

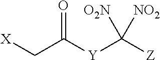

| Com- | | | |
|---|---|---|---|
| pound | | | |
| No. | X | Y | Z |
| III-1 | Br | —N(H)CH₂— | methyl |
| III-2 | Br | —N(H)CH₂— | ethyl |
| III-3 | Br | —N(H)CH₂— | n-pentyl |
| III-4 | Br | —N(H)CH₂— | hydrogen |
| III-5 | Br | —N(H)CH₂CH₂— | methyl |
| III-6 | Br | —N(H)(CH₂)₄— | methyl |
| III-7 | Br | —N(CH₃)CH₂— | methyl |
| III-8 | Br | —N(CH₃)(CH₂)₃— | methyl |
| III-9 | Br | —N(H)C(CH₃)(H)— | methyl |
| III-10 | Br | —N(H)C(CH₃)(H)CH₂— | methyl |
| III-11 | Br | —CH₂— | methyl |
| III-12 | Br | —(CH₂)₂— | methyl |
| III-13 | Br | —CH₂— | ethyl |
| III-14 | Br | —(CH₂)₄— | isopropyl |
| III-15 | Br | —(CH₂)₂— | n-pentyl |
| III-16 | Br | —CH₂— | hydrogen |
| III-17 | Br | —CH₂CH₂C(CH₃)₂— | methyl |
| III-18 | Br | —CH₂C(CH₃)₂CH₂— | methyl |
| III-19 | Cl | —N(H)CH₂— | methyl |
| III-20 | Cl | —N(H)CH₂— | ethyl |
| III-21 | Cl | —N(CH₃)CH₂— | methyl |
| III-22 | Cl | —(CH₂)₂— | methyl |
| III-23 | I | —N(H)CH₂— | methyl |
| III-24 | I | —N(H)CH₂— | ethyl |
| III-25 | I | —N(CH₃)CH₂— | methyl |
| III-26 | I | —(CH₂)₂— | methyl |
| III-27 | —OS(O)₂CH₃ | —N(H)CH₂— | methyl |
| III-28 | —OS(O)₂CH₃ | —N(CH₃)CH₂— | methyl |
| III-29 | —OS(O)₂CF₃ | —N(H)CH₂— | methyl |
| III-30 | —OS(O)₂CF₃ | —N(CH₃)CH₂— | methyl |
| III-31 | 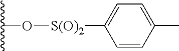 | —N(H)CH₂— | methyl |
| III-32 | 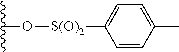 | —N(CH₃)CH₂— | methyl |
| III-33 | —OC(O)CF₃ | —N(H)CH₂— | methyl |
| III-34 | —OC(O)CF₃ | —N(CH₃)CH₂— | methyl |
| III-35 | —OC(O)CF₃ | —(CH₂)₂— | methyl |

Methods for preparing compounds described herein are illustrated in the following synthetic schemes. These schemes are given for the purpose of illustrating the invention, and should not be regarded in any manner as limiting the scope or the spirit of the invention. Starting materials shown in the schemes can be obtained from commercial sources or can be prepared based on procedures described in the literature.

The synthetic route illustrated in Scheme 1 depicts a general method for preparing cyclic geminal di-nitro compounds. In the first step, chloro epoxide A1 is reacted with t-butylamine to provide hydroxy heterocyclic compound B1. Mesylation of the hydroxyl group of heterocyclic compound B1 with methylsulfonyl chloride gives mesylate C1, which upon reacting with NaNO₂ generates cyclic mono-nitro compound D1. Further nitration of compound D1 can be carried out using NaNO₂ in the presence of Na₂S₂O₈ and K₃Fe(CN)₆ to provide geminal di-nitro heterocyclic compound E1. Reacting compound E1 with boron trifluoride etherate and acetyl bromide F provides the desired product G1. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein.

This synthetic procedure illustrated in Scheme 1 and described above is contemplated to be applicable to preparing compounds having various substituents at the R₁, R₂, R₃ and R₄ positions. If a particular epoxide compound embraced by A1 should contain a functional group sensitive to one or more of the synthetic transformations in Scheme 1, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991.

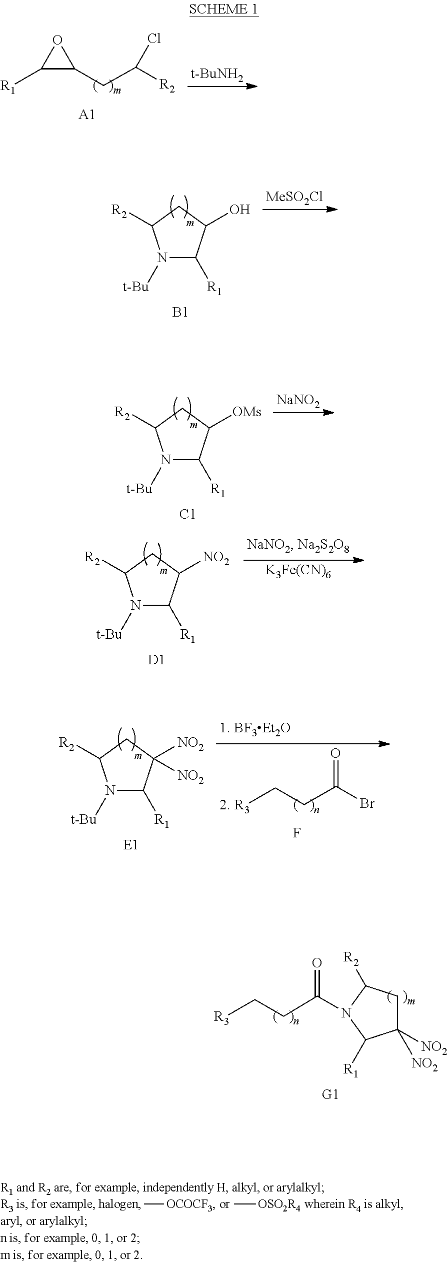

Scheme 2 illustrates a more specific embodiment of the synthetic route shown in Scheme 1 when m is 0. In the first step, epoxide A2 is reacted with t-butylamine to provide hydroxyl azetidine B2. Mesylation of the hydroxyl group of azetidine B2 with methylsulfonyl chloride gives azetidine mesylate C2, which upon reacting with NaNO₂ generates mono-nitro azetidine D2. Further nitration of mono-nitro azetidine D2 with NaNO₂ in the presence of Na₂S₂O₈ and K₃Fe(CN)₆ furnishes the geminal di-nitro azetidine E2. Reaction of azetidine E2 with boron trifluoride etherate and acetyl bromide compound F provides the desired di-nitro azetidine product G2. This synthetic procedure is contemplated to be applicable to preparing compounds having various substituents at the R₁, R₂, R₃ and R₄ positions. If a particular epoxide compound embraced by A2 should contain a functional group sensitive to one or more of the synthetic transformations in Scheme 2, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991. Furthermore, mono-nitro compounds can be prepared by treating mono-nitro compound D2 with a Lewis Acid (e.g., boron trifluoride etherate) and acetyl bromide compound F to provide the desired mono-nitro product.

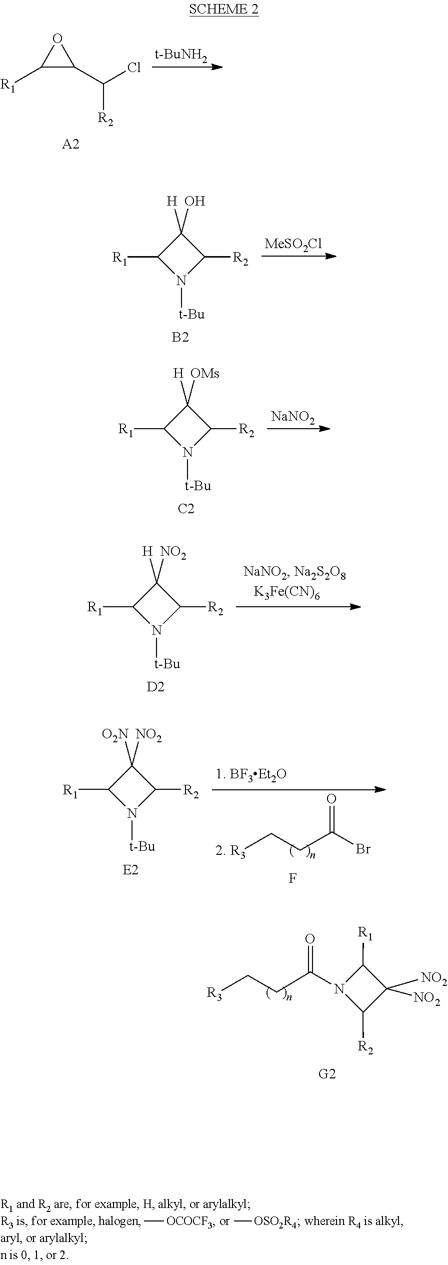

Scheme 3 illustrates another more particular embodiment of the synthetic route shown in Scheme 1 when both R₁ and R₂ are hydrogen and m is 0. In the first step, commercially available epichlorohydrin A3 is reacted with t-butylamine to provide hydroxyl azetidine B3. Mesylation of the hydroxyl group of azetidine B3 with methylsulfonyl chloride gives azetidine mesylate C3, which upon reacting with NaNO₂ generates mono-nitro azetidine D3. Further nitration of mono-nitro azetidine D3 with NaNO₂ in the presence of Na₂S₂O₈ and K₃Fe(CN)₆, furnishes the geminal di-nitro azetidine E3. Reaction of azetidine E3 with boron trifluoride etherate and bromoacetyl bromide provides the desired di-nitro azetidine F3. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. Furthermore, mono-nitro compounds can be prepared by treating mono-nitro compound D3 with a Lewis Acid (e.g., boron trifluoride etherate) and acetyl bromide compound F to provide the desired mono-nitro product.

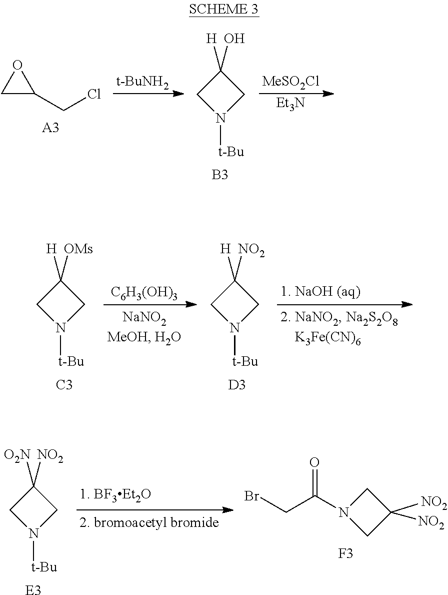

Scheme 4 illustrates an alternative exemplary procedure for preparing cyclic geminal di-nitro compounds. In the first step, heterocyclic compound A4 is reacted with an oxidant, such as pyridinium dichromate (PDC), to provide heterocyclic ketone B4. Reaction of ketone B4 with hydroxylamine gives heterocyclic oxime C4, which upon reaction with N-bromosuccinimide (NBS) produces bromo nitro compound D4. Reaction of compound D4 with NaBH₄ furnishes mono-nitro compound E4. Reaction of mono-nitro compound E4 with NaNO₂ in the presence of Na₂S₂O₈ and K₃Fe(CN)₆ provides geminal di-nitro heterocyclic compound F4. Reaction of compound F4 with a deprotecting agent and acetyl bromide compound F provides the desired cyclic geminal di-nitro product G4. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. Furthermore, mono-nitro compounds can be prepared by treating mono-nitro compound D4 with a deprotecting agent and acetyl bromide compound F to provide the desired mono-nitro product.

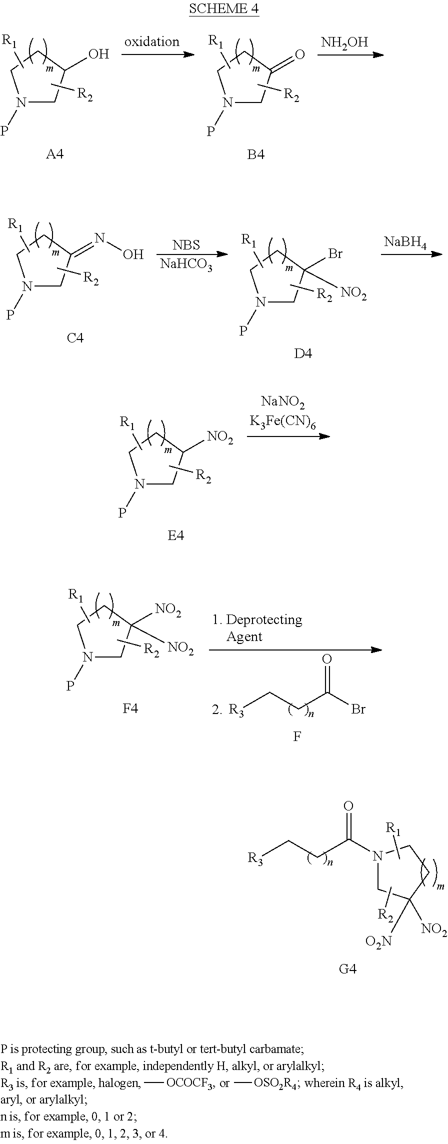

Scheme 5 illustrates yet another exemplary procedure for preparing cyclic geminal di-nitro compounds with initial steps different from those shown in Scheme 4. In the first step, heterocyclic compound A4 is reacted with methylsulfonyl chloride to provide heterocyclic mesylate B5. Reaction of mesylate B5 with NaNO₂ gives mono-nitro compound E4. Nitration of compound E4 with NaNO₂ in the presence of Na₂S₂O₈ and K₃Fe(CN)₆ provides geminal di-nitro compound F4. Reaction of compound F4 with a deprotecting agent and acetyl bromide compound F provides the desired di-nitro product G4. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein.

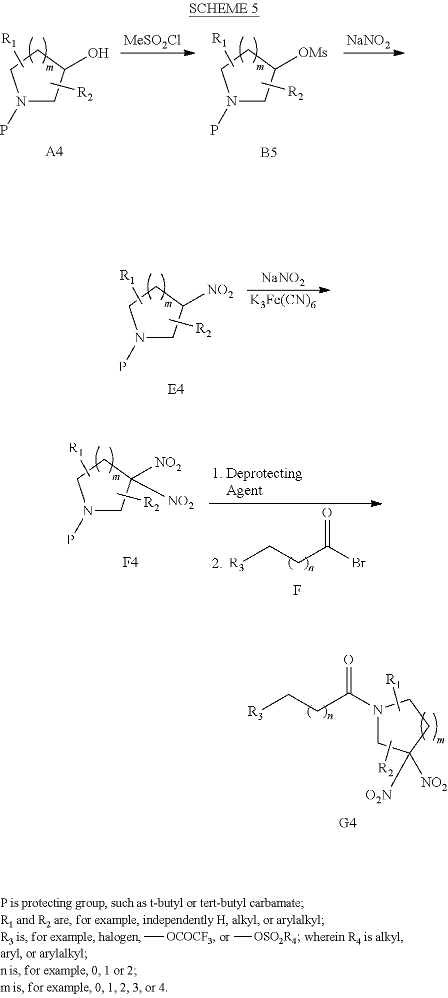

The synthetic route illustrated in Scheme 6 depicts an exemplary method for preparing cyclic vicinal di-nitro compounds. In the first step, cycloalkene A6 is reacted with N₂O₄ to provide vicinal di-nitro compound B6. Reaction of compound B6 with a deprotecting agent and acetyl bromide compound F provides the desired vicinal di-nitro product C6. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. This synthetic procedure is contemplated to be applicable to preparing compounds having various substituents at the R₁, R₂, R₃ and R₄ positions. If a particular cycloalkene compound embraced by A6 should contain a functional group sensitive to one or more of the synthetic transformations in Scheme 6, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991.

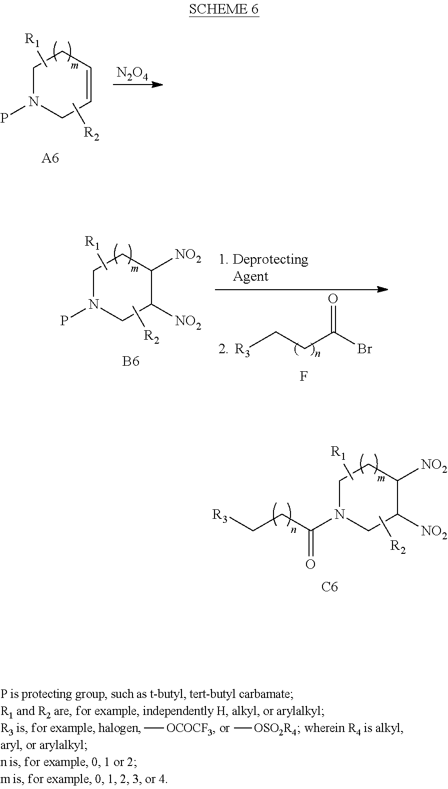

The synthetic route illustrated in Scheme 7 depicts a general method for preparing cyclic mono-nitro compounds. In the first step, chloro epoxide A7 is reacted with t-butylamine to provide hydroxy heterocyclic compound B7. Mesylation of the hydroxyl group of heterocyclic compound B7 with methylsulfonyl chloride gives mesylate C7 which upon reacting with NaNO₂ generates cyclic mono-nitro compound D7. Reaction of compound D7 with boron trifluoride etherate and acetyl bromide F provides the desired product G7. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. This synthetic procedure illustrated in Scheme 7 is contemplated to be applicable to preparing compounds having various substituents at the R₁, R₂, R₃ and R₄ positions. If a particular epoxide compound embraced by A7 should contain a functional group sensitive to one or more of the synthetic transformations in Scheme 7, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991.

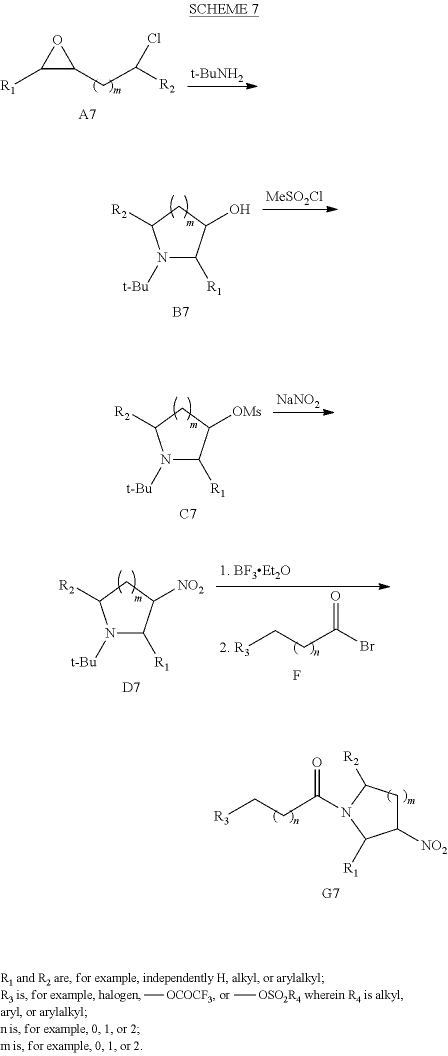

The synthetic routes described above can be modified to prepare compounds having an alkyl halide attached to the ring nitrogen atom. Exemplary synthetic procedures for preparing such compounds include reducing the amide group of compound G1-G4, G7, and C6 to an amine. Alternatively, compound F used in the procedures above could be replaced with an appropriately protected alkylhalide, such that after the alkylation reaction, the protected alkyl group attached to the ring nitrogen atom is deprotected and converted to an alkyl chloride or bromide.

Scheme 8 depicts another exemplary method for preparing cyclic mono-nitro and di-nitro compounds. Reaction of ketone B8 with hydroxylamine gives heterocyclic hydroxylamine C8, which upon reaction with N-bromosuccinimide (NBS) produces bromo nitro compound D8. Reaction of compound D8 with NaBH₄ furnishes mono-nitro compound E8. The hydroxyl protecting group (P, which may be, for example, a tert-butyldimethylsilyl group) and the 1,2-dihydroxyethane protecting group are removed using standard deprotection conditions. Exemplary deprotection conditions for removing a tert-butyldimethyl silyl group include addition of tetra-n-butylammonium fluoride. Exemplary deprotection conditions for removing a 1,2-dihydroxyethane protecting group include addition of hydrochloric acid and water. Hydroxy-ketone F8 can be converted to α-bromo ketone G8 by first reacting compound F8 with methanesulfonyl chloride to form a mesylate and then adding sodium bromide to form α-bromo ketone G8.

Di-nitro compounds can be prepared by reacting mono-nitro compound E8 with NaNO₂ in the presence of Na₂S₂O₈ and K₃Fe(CN)₆ to provide geminal di-nitro heterocyclic compound H8. The hydroxyl protecting group (P, which may be, for example, a tert-butyldiimethyl silyl group) and the 1,2-dihydroxyethane protecting group of compound H8 may be removed using standard deprotection conditions. Exemplary deprotection conditions for removing a tert-butyldiimethyl silyl group include addition of tetra-n-butylammonium fluoride. Exemplary deprotection conditions for removing a 1,2-dihydroxyethane protecting group include addition of hydrochloric acid and water. Hydroxy-ketone I8 can be converted to α-bromo ketone J8 by first reacting compound I8 with methanesulfonyl chloride to form a mesylate and then adding sodium bromide to form α-bromo ketone J8. Further description of related synthetic procedures are described in, for example, Archibald et al. in J. Org. Chem. 1990, 55, 2920-2924 and J. P. Agrawal, R. D. Hodgson, Organic Chemistry of Explosives, Wiley & Sons, England, 2007 and references cited therein.

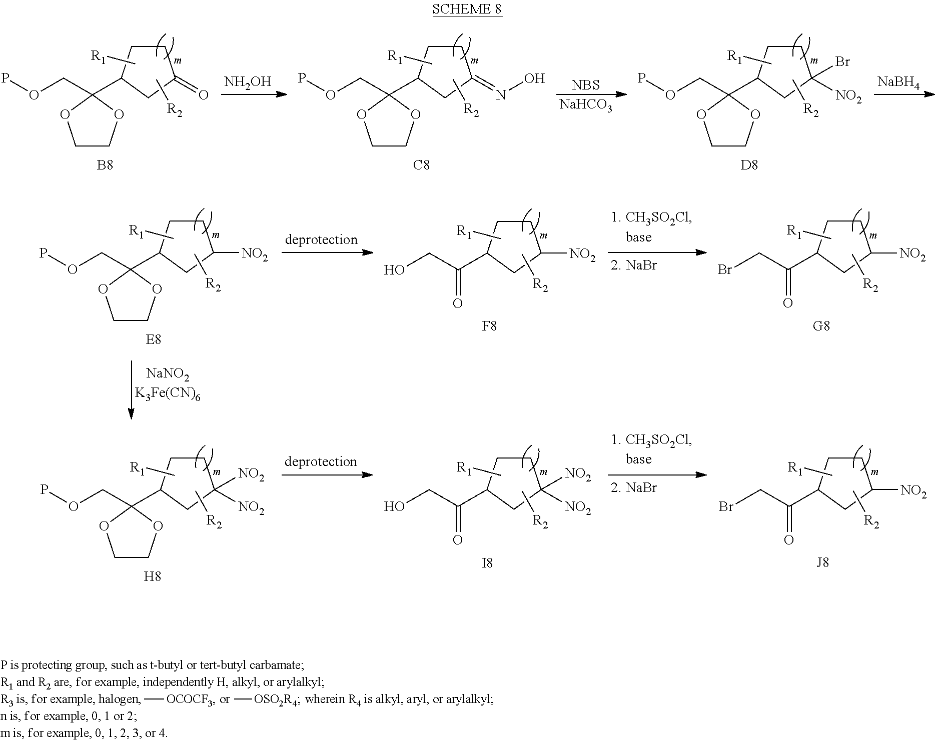

Scheme 9 illustrates an exemplary procedure for preparing acyclic geminal di-nitro compounds. In the first step, protected amino alcohol A9 is reacted with methylsulfonyl chloride to provide mesylate B9. Reaction of mesylate B9 with NaNO₂ gives mono-nitro compound E9. Nitration of compound E9 with NaNO₂ in the presence of Na₂S₂O₈ and K₃Fe(CN)₆ provides geminal di-nitro compound F9. Reaction of compound F9 with a deprotecting agent and acetyl bromide compound F provides the desired di-nitro product G9. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein.

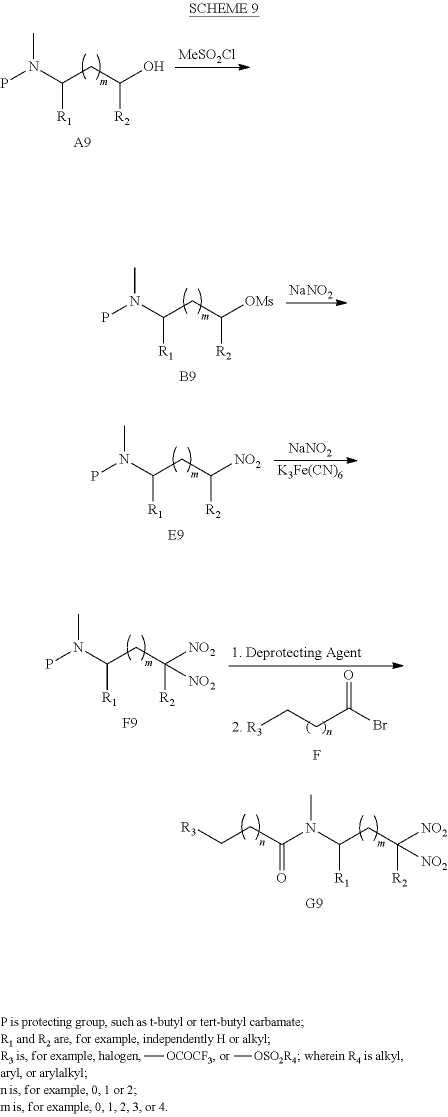

### III. METHODS OF TREATING NON-HODGKIN'S LYMPHOMA AND CERTAIN LEUKEMIAS USING ORGANONITRO COMPOUNDS

The invention provides methods of treating non-Hodgkin's lymphoma and certain leukemias. Exemplary leukemias contemplated for treatment using the organonitro compounds described herein include chronic lymphocytic leukemia, acute myelogenous leukemia, chronic myelogenous leukemia, and acute lymphoblastic leukemia. Treatment methods include the use of the organonitro compounds described herein as stand-alone chemotherapeutic agents and/or as radiation sensitizers. Although not wishing to be bound by a particular theory, it is understood that organonitro compounds described herein can release reactive free radicals that are cytotoxic to leukemia cells and non-Hodgkin lymphoma cells.

### Methods of Treating Medical Disorders

One aspect of the invention provides a method of treating a disorder selected from the group consisting of non-Hodgkin's lymphoma, chronic lymphocytic leukemia, acute myelogenous leukemia, chronic myelogenous leukemia, and acute lymphoblastic leukemia. The method comprises administering to a patient in need thereof a therapeutically effective amount of an organonitro compound described herein, such as a compound of Formula I or II, which as described above, Formula I is represented by:

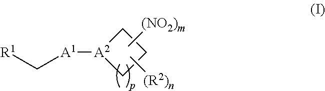

or a pharmaceutically acceptable salt or solvate thereof, wherein:

A¹ is —C(O)— or —(C(R³)₂)ₓC(O)(C(R³)₂)ₓ—;

A² is N or —C(R⁴)—;

R¹ is halogen, —OS(O)₂R⁵, or —OC(O)CF₃;

R² is C₁-C₆alkyl;

R³ and R⁴ each represent independently for each occurrence hydrogen or C₁-C₅alkyl;

R⁵ is C₁-C₆alkyl, C₁-C₆haloalkyl, aryl, or aralkyl;

m and p are independently 1, 2, or 3; and

n and x each represent independently for each occurrence 0, 1, 2, or 3; and

Formula II is represented by:

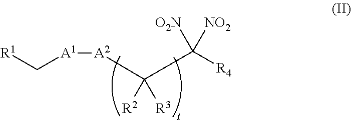

or a pharmaceutically acceptable salt or solvate thereof: wherein:

A¹ is —C(O)— or —(C(R⁵)₂)ₓC(O)(C(R⁵)₂)ₓ—;

A² is —N(R⁵)— or —C(R²)(R³)—;

R¹ is halogen, —OS(O)₂R⁶, or —OC(O)CF₃;

R² and R³ each represent independently for each occurrence hydrogen or C₁-C₆alkyl; or R² and R³ are taken together with the carbon atom to which they are attached to form a 3-6 membered, saturated carbocyclic ring;

R⁴ is hydrogen or C₁-C₆alkyl;

R⁵ represents independently for each occurrence hydrogen or C₁-C₆alkyl;

R⁶ is C₁-C₆alkyl, C₁-C₆haloalkyl, aryl, or aralkyl;

t is an integer in the range from 1 to 12; and

x represents independently for each occurrence 0, 1, 2, or 3.

In certain embodiments, the disorder is non-Hodgkin's lymphoma.

In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma.

In certain embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

In certain embodiments, the disorder is chronic lymphocytic leukemia, acute myelogenous leukemia, chronic myelogenous leukemia, or acute lymphoblastic leukemia.

In certain embodiments, the patient is a human.

In certain embodiments, the compound is one of the generic or specific compounds described in Section II, such as a compound of Formula I, a compound embraced by one of the further embodiments describing definitions for certain variables of Formula I, a compound of Formula I-A, or a compound embraced by one of the further embodiments describing definitions for certain variables of Formula I-A.

For example, with regards to Formula I, in certain embodiments, the compound corresponds to Formula I where A¹ is —C(O)—. In certain other embodiments, A¹ is —(C(R³)₂)ₓC(O)(C(R³)₂)ₓ—. In certain other embodiments, A¹ is —C(O)(C(R³)₂)ₓ—.

In certain embodiments, A² is N. In certain other embodiments, A² is —C(R⁴)—.

In certain embodiments, R¹ is halogen, —OS(O)₂R⁵, or —OC(O)CF₃. In certain other embodiments, R¹ is halogen. In certain other embodiments, R¹ is —OS(O)₂R⁵. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain embodiments, R¹ is bromo.

In certain embodiments, m is 2. In certain other embodiments, m is 1.

In certain embodiments, n is 0. In certain other embodiments, n is 1. In certain other embodiments, n is 2.

In certain embodiments, p is 1. In certain other embodiments, p is 2. In certain other embodiments, p is 3.

In certain embodiments, the compound is a compound of Formula I-A:

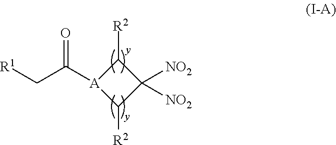

or a pharmaceutically acceptable salt or solvate thereof, wherein:

A is N or C(H);

R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;

R² represents independently for each occurrence hydrogen or methyl; and

y represents independently for each occurrence 1 or 2.

In certain embodiments, A is N. In certain other embodiments, A is C(H).

In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² is hydrogen or methyl. In certain embodiments, R² is hydrogen.

In certain embodiments, y is 1. In certain embodiments, one occurrence of y is 1, and the other occurrence of y is 2. In certain other embodiments, y is 2.

In certain embodiments, the compound is

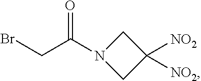

or a pharmaceutically acceptable salt or solvate thereof. In certain other embodiments, the compound is

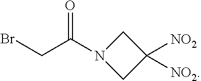

In certain other embodiments, the compound is one of the compounds listed in Tables 1 and 2 herein or a pharmaceutically acceptable salt or solvate thereof.

The description above describes multiple embodiments relating to methods of treating various disorders using certain organonitro compounds. The patent application specifically contemplates all combinations of the embodiments. For example, the invention contemplates treating non-Hodgkin's lymphoma and certain leukemias by administering a therapeutically effective amount of a compound of Formula I-A wherein A is N, R¹ is chloro or bromo, and R² is hydrogen. Further, for example, the invention contemplates treating non-Hodgkin's lymphoma and certain leukemias by administering a therapeutically effective amount of a compound of Formula II wherein A¹ is —C(O)—, A² is N(R⁵), and R² and R³ are hydrogen. Further, in certain embodiments, the disorder is a non-Hodgkin's lymphoma selected from the group consisting of a diffuse large B-cell lymphoma and follicular lymphoma.

In certain embodiments, the compound is used as a chemotherapeutic agent. For example, the compound may be used as a stand-alone chemotherapeutic agent or used in combination with other chemotherapeutic agents.

In certain other embodiments, the compound is used as a radiation sensitizer.

### Combination Therapy

Another aspect of the invention embraces combination therapy, which includes the administration of an organonitro compound described herein (such as compound of Formula I or II) and a second agent as part of a specific treatment regimen intended to provide the beneficial effect from the co-action of these therapeutic agents. The beneficial effect of the combination may include pharmacokinetic or pharmacodynamic co-action resulting from the combination of therapeutic agents. Administration of these therapeutic agents in combination typically is carried out over a defined time period (e.g., hours or days depending upon the combination selected). The combination therapy may involve administration of two or more of these therapeutic agents as part of separate monotherapy regimens that result in the combinations of the present invention. Combination therapy also includes administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues.

It is understood that the therapeutic agents can be administered by the same route or by different routes. For example, a first therapeutic agent of the combination selected may be administered by pulmonary administration while the other therapeutic agent(s) of the combination may be administered orally. Alternatively, for example, all therapeutic agents may be administered orally or all therapeutic agents may be administered by pulmonary administration.

Accordingly, in certain embodiments, one or more of the methods described herein above further comprise administering to the patient a therapeutically effective amount of a second therapeutic agent. In certain embodiments, the second therapeutic agent is, for example, adenosine, an antimicrobial compound, an aldosterone antagonist, an alpha-adrenergic receptor antagonist, a β-adrenergic agonist, an anti-allergic compound, an anti-diabetic compound, an anti-hyperlipidemic drug, an anti-tussive compound, an angiotensin II antagonist, an angiotensin-converting enzyme (ACE) inhibitor, an antioxidant, an antithrombotic, a vasodilator drug, a β-adrenergic antagonist, a bronchodilator, a calcium channel blocker, a diuretic, an endothelin antagonist, an expectorant, a hydralazine compound, a H2-receptor antagonist, a neutral endopeptidase inhibitor, a nonsteroidal antiinflammatory compound (NSAID), a phosphodiesterase inhibitor, a potassium channel blocker, a platelet reducing agent, a proton pump inhibitor, a renin inhibitor, a selective cyclooxygenase-2 (COX-2) inhibitor, a steroid. In certain other embodiments, the second therapeutic agent is selected from the group consisting of an antimicrobial compound, a β-adrenergic agonist, an anti-allergic compound, an anti-tussive compound, an antioxidant, a bronchodilator, an expectorant, a nonsteroidal antiinflammatory compound (NSAID), a phosphodiesterase inhibitor, a selective cyclooxygenase-2 (COX-2) inhibitor, and a steroid.

### IV. PHARMACEUTICAL COMPOSITIONS FOR TREATING NON-HODGKIN'S LYMPHOMA AND CERTAIN LEUKEMIAS USING ORGANONITRO COMPOUNDS

The invention provides pharmaceutical compositions comprising an organonitro compound described herein, such as a compound of Formula I or II, for use in treating non-Hodgkin's lymphoma and certain leukemias. In certain embodiments, the pharmaceutical compositions preferably comprise a therapeutically-effective amount of one or more of the organonitro compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders a compound of the present invention orally bioavailable.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug administered by subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Organonitro compounds and/or pharmaceutical compositions thereof may also be administered directly to the lung by inhalation. For administration by inhalation, organonitro compounds and/or pharmaceutical compositions thereof may be conveniently delivered to the lung by a number of different devices. For example, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver organonitro compounds and/or pharmaceutical compositions thereof directly to the lung.

Alternatively, a Dry Powder Inhaler (“DPI”) device may be used to administer an organonitro compound and/or pharmaceutical composition thereof to the lung. DPI devices typically use a mechanism such as a burst of gas to create a cloud of dry powder inside a container, which may then be inhaled by the patient, and are well known in the art. A popular variation is the multiple dose DPI (“MDDPI”) system, which allows for the delivery of more than one therapeutic dose. MDDPI devices are commercially available from a number of pharmaceutical companies (e.g., Schering Plough, Madison, N.J.). For example, capsules and cartridges of gelatin for use in an inhaler or insufflator may be formulated containing a powder mix of an organonitro compound and/or pharmaceutical composition thereof and a suitable powder base such as lactose or starch for these systems.

Another type of device that may be used to deliver a cyclic nitro compound and/or pharmaceutical composition thereof to the lung is a liquid spray device supplied, for example, by Aradigm Corporation, Hayward, Calif. Liquid spray systems use extremely small nozzle holes to aerosolize liquid drug formulations that may then be directly inhaled into the lung.

In some embodiments, a nebulizer is used to deliver an organonitro compound and/or pharmaceutical composition thereof to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96). Examples of nebulizers include devices supplied by Sheffield Pharmaceuticals, St. Louis, Mo. (see, e.g., Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, Ohio.

In other embodiments, an electrohydrodynamic (“EHD”) aerosol device is used to deliver an organonitro compound and/or pharmaceutical composition thereof to the lung of a patient. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering an organonitro compound and/or pharmaceutical composition thereof to the lung with an EHD aerosol device and such optimization is routinely performed by one of skill in the art. EHD aerosol devices may more efficiently deliver drugs to the lung than existing pulmonary delivery technologies.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrase “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

### V. KITS FOR USE IN MEDICAL APPLICATIONS

Another aspect of the invention provides a kit for treating a disorder. The kit comprises: i) instructions for treating a disorder described herein, such as a disorder selected from the group consisting of non-Hodgkin's lymphoma, chronic lymphocytic leukemia, acute myelogenous leukemia, chronic myelogenous leukemia, and acute lymphoblastic leukemia; and ii) an organonitro compound described herein, such as a compound of Formula I or II. The kit may comprise one or more unit dosage forms containing an amount of organonitro compound described herein, such as a compound of Formula I or II, that is effective for treating said disorder.

In certain embodiments, the disorder is non-Hodgkin's lymphoma. In certain embodiments, the disorder is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the disorder is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

In certain other embodiments, the disorder is leukemia. In certain embodiments, the disorder is chronic lymphocytic leukemia, acute myelogenous leukemia, chronic myelogenous leukemia, or acute lymphoblastic leukemia.

In certain embodiments, the organonitro compound is one of the generic or specific compounds described in Section II, such as a compound of Formula I, a compound embraced by one of the further embodiments describing definitions for certain variables of Formula I, a compound of Formula I-A, or a compound embraced by one of the further embodiments describing definitions for certain variables of Formula I-A. In certain embodiments, the compound is a compound of Formula II, a compound embraced by one of the further embodiments describing definitions for certain variables of Formula II, a compound of Formula II-A, or a compound embraced by one of the further embodiments describing definitions for certain variables of Formula II-A.

The description above describes multiple aspects and embodiments of the invention, including organonitro compounds, compositions comprising organonitro compounds, methods of using the organonitro compounds, and kits. The patent application specifically contemplates all combinations and permutations of the aspects and embodiments. For example, the invention contemplates treating non-Hodgkin's lymphoma and certain leukemias in a human patient by administering a therapeutically effective amount of a compound of Formula I-A. Further, for example, the invention contemplates a kit for treating non-Hodgkin's lymphoma and certain leukemias, the kit comprising instructions for treating non-Hodgkin's lymphoma and certain leukemias, and ii) an organonitro compound described herein, such as a compound of Formula I.

### EXAMPLES

The invention now being generally described, will be more readily understood by reference to the following examples, which are included merely for purposes of illustration of certain aspects and embodiments of the present invention, and are not intended to limit the invention.

### Example 1

The ability of 1-bromoacetyl-3,3-dinitroazetidine (ABDNAZ) to induce apoptosis of B-cell lymphoma Daudi cells was evaluated according to the procedures described in Part I below. Results of the experimental assay are described in Part II below.

### Part I: Procedures

B-cell lymphoma Daudi cells were grown and maintained in Dulbecco's modified essential medium (DMEM) (INVITROGEN™, Grand Island, N.Y.) supplemented with 10% fetal calf serum, 100 units/mL penicillin, and 100 μg/mL streptomycin in a 37° C. humidified incubator with a mixture of 95% air and 5% CO₂. Experiments were performed on exponentially growing cells with a cell population doubling time of approximately 20-36 hours.

After exposing the B-cell lymphoma Daudi cells to ABDNAZ, apoptotic cell death was quantitated by measuring the number of subdiploid (sub-G1) cells stained with propidium iodide (PI), according to a procedure described previously by Ning and coworkers. Ning, S., and Knox, S. J. in Int. J. Radiat. Oncol. Biol. Phys. (2004) 60:197-203. Briefly, ABDNAZ-treated and untreated control cells were collected and fixed with cold 100% ethanol. Cells were pelleted, treated with 100 μg/mL of RNase A in PBS-EDTA solution for 30 min and stained with 50 μg/mL of PI for 10 min at room temperature. The DNA content was analyzed with a FACScan flowcytometer (Becton Dickison, San Jose, Calif.). The percentage of cells in the sub-G1 phase was calculated as described previously by Ning and coworkers.

To examine whether ABDNAZ-induced apoptotic cell death was due to the generation of reactive oxygen species (ROS), Daudi cells were treated with a glutathione (GSH) precursor, N-acetylcysteine (NAC), and a GSH synthesis inhibitor, buthionine-(S,R)-sulfoxime (BSO), and the level of apoptosis was measured.

### Part II: Results

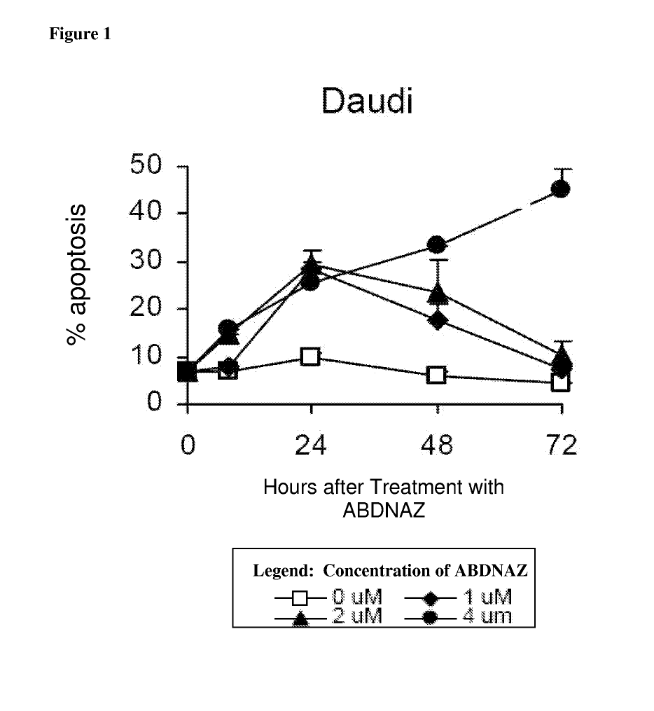

As shown in FIG. 1, Daudi cell apoptosis increased to 45% when measured at 72 hours after exposing the Daudi cells to a 4 mM concentration of ABDNAZ. Exposure of Daudi cells to ABDNAZ at a concentration of 2 mM caused Daudi cell apoptosis in the amount of 30% when measured at 24 hours.

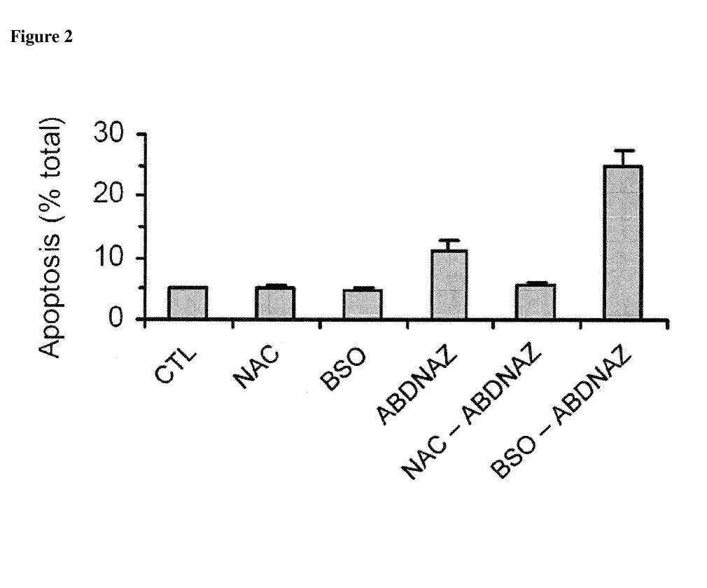

Referring to FIG. 2, pre-treatment of the Daudi cells with N-acetylcysteine decreased ABDNAZ-induced apoptosis from 11% to 5%. In contrast, pre-treatment of the Daudi cells with buthionine-(S,R)-sulfoxime increased ABDNAZ-induced apoptosis from 11% to 25%, suggesting that intracellular redox status and ROS generation may be important for ABDNAZ-induced apoptotic cell death. The abbreviation CTL in FIG. 2 indicates a control experiment.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating a disorder selected from the group consisting of non-Hodgkin's lymphoma, chronic lymphocytic leukemia, acute myelogenous leukemia, chronic myelogenous leukemia, and acute lymphoblastic leukemia, comprising administering to a patient in need thereof a therapeutically effective amount of a compound of Formula I or II, wherein Formula I is represented by: 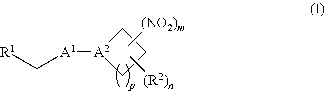
or a pharmaceutically acceptable salt or solvate thereof, wherein:
A¹ is —C(O)— or —(C(R³)₂)ₓC(O)(C(R³)₂)ₓ—;
A² is N or —C(R⁴)—;
R¹ is halogen, —OS(O)₂R⁵, or —OC(O)CF₃;
R² is C₁-C₆alkyl;
R³ and R⁴ each represent independently for each occurrence hydrogen or C₁-C₅alkyl;
R⁵ is C₁-C₆alkyl, C₁-C₆haloalkyl, aryl, or aralkyl;
m and p are independently 1, 2, or 3; and
n and x each represent independently for each occurrence 0, 1, 2, or 3; and Formula II is represented by: 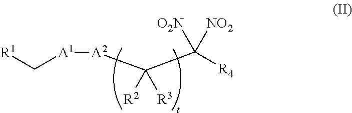
or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—;
R¹ is halogen, —OS(O)₂R⁶, or —OC(O)CF₃;
R² and R³ each represent independently for each occurrence hydrogen or C₁-C₆alkyl; or R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
R⁶ is C₁-C₆alkyl, C₁-C₆haloalkyl, aryl, or aralkyl;
t is an integer in the range from 1 to 12; and
x represents independently for each occurrence 0, 1, 2, or 3.

2. The method of claim 1, wherein the disorder is non-Hodgkin's lymphoma.

3. The method of claim 1, wherein the disorder is chronic lymphocytic leukemia, acute myelogenous leukemia, chronic myelogenous leukemia, or acute lymphoblastic leukemia.

4. The method of claim 1, wherein the patient is a human.

5. A kit for treating a disorder, comprising:
i) instructions for treating a disorder selected from the group consisting of non-Hodgkin's lymphoma, chronic lymphocytic leukemia, acute myelogenous leukemia, chronic myelogenous leukemia, and acute lymphoblastic leukemia; and
ii) a compound of Formula I or II, wherein Formula I represented by: 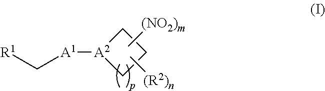
or a pharmaceutically acceptable salt or solvate thereof, wherein:
A¹ is —C(O)— or —(C(R³)₂)ₓC(O)(C(R³)₂)ₓ—;
A² is N or —C(R⁴)—;
R¹ is halogen, —OS(O)₂R⁵, or —OC(O)CF₃;
R² is C₁-C₆alkyl;
R³ and R⁴ each represent independently for each occurrence hydrogen or C₁-C₅alkyl;
R⁵ is C₁-C₆alkyl, C₁-C₆haloalkyl, aryl, or aralkyl;
m and p are independently 1, 2, or 3; and
n and x each represent independently for each occurrence 0, 1, 2, or 3; and Formula II is represented by: 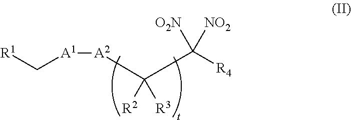
or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—;
R¹ is halogen, —OS(O)₂R⁶, or —OC(O)CF₃;
R² and R³ each represent independently for each occurrence hydrogen or C₁-C₆alkyl; or R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
R⁶ is C₁-C₆alkyl, C₁-C₆haloalkyl, aryl, or aralkyl;
t is an integer in the range from 1 to 12; and
x represents independently for each occurrence 0, 1, 2, or 3.

6. The method of claim 1, wherein said compound is a compound of Formula I.

7. The method of claim 6, wherein A¹ is —C(O)—.

8. The method of claim 7, wherein A² is N.

9. The method of claim 8, wherein R¹ is chloro, bromo, —OS(O)₂-(para-methylphenyl), —OS(O)₂CH₃, —OS(O)₂CF₃, or —OC(O)CF₃.

10. The method of claim 8, wherein R¹ is bromo.

11. The method of claim 10, wherein m is 2.

12. The method of claim 11, wherein n is

13. The method of claim 12, wherein p is 1.

14. The method of claim 6, wherein the compound is a compound of Formula I-A: 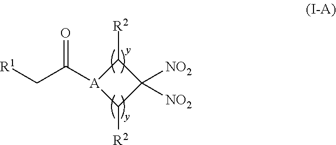
or a pharmaceutically acceptable salt or solvate thereof, wherein:
A is N or C(H);
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R² represents independently for each occurrence hydrogen or methyl;
y represents independently for each occurrence 1 or 2.

15. The method of claim 14, wherein A is N.

16. The method of claim 15, wherein R¹ is bromo.

17. The method of claim 16, wherein y is 1.

18. The method of claim 1, wherein said compound is a compound of Formula II.

19. The method of claim 1, wherein said compound is one of the compounds in Tables 1-3, or a pharmaceutically acceptable salt or solvate thereof.

20. The method of claim 1, wherein said compound is 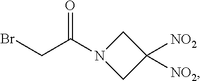
or a pharmaceutically acceptable salt or solvate thereof.

21. The method of claim 1, wherein said compound is [chemical structure]
